(19) United States
(12) Patent Application Publication
(10) Pub. No.: US 2002/0182306 A1
(43) Pub. Date: Dec. 5, 2002

(54) Coating system

(75) Inventors: Roger Berlin, Mendham, NJ (US); Justin Bianco, Bowling Green, VA (US)

Correspondence Address: DARBY & DARBY P.C., P.O. Box 5257, New York, NY, 10150-5257, US

(73) Assignee: AMERICAN HOME PRODUCTS CORPORATION

(21) Appl. No.: 10/171,054

(22) Filed: Jun. 12, 2002

Related U.S. Application Data
(63) Continuation of application No. 09/538,583, filed on Mar. 29, 2000, now abandoned.
(60) Provisional application No. 60/126,692, filed on Mar. 29, 1999.

(51) Int. Cl. B05D 1/36; A61L 2/00

(52) U.S. Cl. 427/2.1

(57) ABSTRACT

The present invention provides a coating system for solid pharmaceutical dosage units, such as tablets. The coating system comprising (a) a subcoat; (b) an optional colorcoat; and (c) a polish coat. In a preferred embodiment, the present invention provides (a) a subcoat comprising (i) hydroxypropyl methylcellulose (HPMC) and (ii) a polysaccharide; (b) a colorcoat comprising (i) a colorant and (ii) a polysaccharide which may be the same as or different from the polysaccharide of the subcoat; and (c) a polish coat comprising (i) a polyethylene glycol and, optionally, (ii) a wax. Also disclosed is a method for providing gloss to a coated pharmaceutical solid dosage unit by coating a pharmaceutical core with the aforementioned polish coat.

### FIELD OF THE INVENTION

The present invention relates to coating systems for solid pharmaceutical dosage units, such as tablets. The present invention further relates to solid pharmaceutical dosage units coated with such coating systems.

### BACKGROUND OF THE INVENTION

Coatings for solid pharmaceutical dosage units serve many purposes. They act to isolate the active pharmaceutical material from the atmosphere, thereby inhibiting its degradation. They further act to isolate the active material from the oral mucosa during ingestion, thereby masking the taste of the pharmaceutically active materials, many of which are foul tasting. Finally, such coatings are useful in improving the visual appearance of the dosage units. This is desirable in view of the often raw, porous appearance of many solid dosage units, such as those produced using compression technology.

Many types of coating technology have heretofore been employed. Most are less than perfect. For instance, films produced from hydroxypropyl methylcellulose exhibit excellent dissolution properties and adequately taste-mask the active material. However, the film itself exhibits an unpleasant taste. Further, tablets coated with such films often exhibit an inelegant appearance.

Sugar-based coatings are often employed in the coating of tablets. Such coatings exhibit excellent taste, taste-masking and appearance qualities. However, relative to film coatings, they are more expensive to produce and slower to dissolve.

### OBJECTS OF THE PRESENT INVENTION

It is therefore an object of the present invention to produce a coating system for solid pharmaceutical dosage units which exhibits good dissolution, appearance, taste and taste-masking properties.

It is further an object of the invention to produce coated solid pharmaceutical dosage units which exhibit good dissolution, appearance, taste and taste-masking properties.

It is still further an object of the present invention to provide a method for improving the luster of a coated dosage unit through the application of a polyethylene glycol-containing polish coat.

These and other objects of the present invention will become apparent from the following description.

### SUMMARY OF THE INVENTION

The present invention is directed to coating systems for solid pharmaceutical dosage units, such as tablets. The present invention is further directed to solid pharmaceutical dosage units coated with such coating systems.

The present invention provides a coating system comprising (a) a subcoat; (b) an optional colorcoat; and (c) a polish coat, as well as solid dosage units bearing such coatings.

In a preferred embodiment, the present invention provides (a) a subcoat comprising (i) hydroxypropyl methylcellulose (HPMC) and (ii) a sugar such as a polysaccharide; (b) a colorcoat comprising (i) a colorant and (ii) a polysaccharide which may be the same as or different from the polysaccharide of the subcoat; and (c) a polish coat comprising (i) a polyethylene glycol and, optionally, (ii) a wax.

In another embodiment, the present invention provides a dosage unit form comprising a medicament-containing core coated with the coating system of the present invention.

In still another embodiment of the present invention there is provided a method for preparing a pharmaceutical solid dosage unit by coating a pharmaceutical core with the aforementioned coating system.

### DETAILED DESCRIPTION OF THE INVENTION

The coating systems of the present invention include at least two (2) coats—a subcoat and a polish coat.

The subcoat comprises a cellulosic material capable of being hydrated and applied as an aqueous solution through film-coating technology. Such materials are well known in the art. Preferred cellulosic materials are hydroxypropyl cellulose (HPC) and hydroxypropyl methylcellulose (HPMC). Most preferred are HPMC's having molecular weights ranging from about 5,000 to about 15,000. Mixtures of such HPMC's may also be employed. HPMC's having molecular weights of 5,000 and 15,000 are referred herein as E5 and HPMC E15, respectively, and are available as Methocel® E5 and Methocel® E15, respectively, from Dow Chemical Co. of Midland, Michigan. Preferably, these HPMC's are present in a weight ratio of HPMC E5:HPMC E15 of about 3:1 to 1:2. Most preferably, these HPMC's are present in a weight ratio of about 2: 1, on the same basis.

The subcoat further contains a sugar. The use of sugars in combination with a cellulosic material is highly attractive from an economic point of view. For example, sucrose is approximately 50 times less expensive than some HPMC's on a per weight basis. The sugar component further acts to taste-mask the unpleasant taste normally associated with the cellulosic material. Useful in the practice of the present invention are simple sugars such as glucose, fructose and mannose and polysaccharides such as sucrose. Preferred are polysaccharides such as sucrose. The use of sucrose is especially preferred.

The subcoat may also include additional materials typical in pharmaceutical coatings. These include such materials as additional sweeteners, antioxidants, plasticizers, flavorants and combinations thereof. In the practice of the present invention, additional materials such as the following are used: acesulfame-K (a sweetener marketed under the tradename Sunett® by Hoechst Celanese Corporation of Portsmouth, Va.), propyl gallate (an antioxidant), and triacetin (a plasticizer).

The subcoat typically includes from about 40 to about 85 percent by weight of cellulosic material and from about 15 to about 60 percent by weight of the sugar component, based upon 100 percent by weight of the subcoat. Preferably, the subcoat includes about 50 to about 70 percent by weight of cellulosic material and about 30 to about 50 percent by weight of the sugar component, on the same basis. Most preferably, the subcoat includes about 60 to about 70 percent by weight of cellulosic material and about 30 to about 40 percent by weight of the sugar component, on the same basis. When the preferred cellulosic material, HPMC, is employed, the subcoat most preferably includes about 60 to about 70 percent by weight of cellulosic material and about 30 to about 40 percent by weight of the sugar component, on the same basis. Most preferably, the subcoat comprises HPMC and sucrose in a 2:1 weight ratio or 67% HPMC and 33% sucrose (on a weight basis).

The polish coat employed in the practice of the present invention comprises a polyethylene glycol (PEG) and, optionally, a wax. The polish coat imparts an elegant, glossy sheen to tablets so coated. In addition to having utility in conjunction with the specific subcoat disclosed herein, the polish coat may generally be utilized on any coated tablet. So long as the polish coat does not adversely interact with the underlying coating, it may be utilized. It is therefore suitable for use on tablets or other solid dosage units which bear film coated or sugar coated layers.

Generally useful as PEG's in the formation of a polish coat are those which readily form aqueous solutions. Generally, these are PEG's having molecular weights ranging from about 500 to about 50,000. Preferably, PEG's should have molecular weights ranging from about 4000 to about 15,000. Most preferred in the practice of the present invention is the use of a PEG having a molecular weight of about 8,000. The PEG-containing polish coats are applied through spray coating technology which is well known in the art. The polish coat may be present in amounts such that they contribute to the weight gain of the final dosage unit from about 0.01 to about 2.0 wt. %, preferably about 0.1 to about 0.5 wt. %, and most preferably about 0.25 wt. %.

The polish coat may additionally contain a wax. Preferably, the optional wax coating is utilized. Most preferably, Carnauba wax is employed. The wax is applied as a dusting to the dosage units to which the PEG-containing polish coating has been applied. The wax is present in extremely small quantities as it is only present to impart additional sheen to the dosage unit. Preferred is the use of the wax coating in amounts of about 0.03 wt. %.

In the practice of the present invention, an optional colorcoat may be present. This coating serves to impart both color and additional layers of taste-masking/degradation protection to the coated dosage units. The colorcoat comprises a colorant and a sugar. Any colorant may be used so long as it is compatible with the subcoat, polish coat and the components thereof. Generally speaking, any pharmaceutically acceptable colorant may be used. A preferred colorant is Opadry®, available from Colorcon of West Point, Pa.

The sugar component used in the colorcoat may be the same as or different from the sugar component used in the formation of the subcoat. Preferably, a polysaccharide is used as the sugar in the colorcoat. Most preferably, sucrose is employed. The colorcoat may also include additional sweeteners, flavorants, or a combination thereof. A preferred additional sweetener is acesulfame-K.

The optional colorcoat may contain up to 60 percent by weight of the sugar component. Typically the colorcoat includes from about 40 to about 60 percent by weight of colorant and from about 40 to about 60 percent by weight of sugar component, based upon 100 percent by weight of the colorcoat. Preferably, the colorcoat includes about 50 percent by weight of colorant and about 50 percent by weight of sugar component, on the same basis.

The coating system of the present invention may be used to coat pharmaceutical cores, such as, for example, cores of dosage unit forms such as, for example, tablets or caplets.

Such cores may include a medicament, such as, for example, ibuprofen, ketoprofen, aspirin, acetaminophen, and the like. Such cores may further comprise the typical excipients found in pharmaceutical dosage units (e.g. disintegrants, antioxidants and sustained-release components).

The coating systems of the present invention may be applied to pharmaceutical cores by methods well known to those skilled in the art such as spray coating. Typically, the above-described layers are applied sequentially. Preferably, the subcoat is applied as about a 12 percent solution and imparts about a 2 percent weight gain, based upon 100 percent by weight of the core. The colorcoat, if employed, is preferably applied as about a 12 percent solution and imparts about a 4 percent by weight gain, based upon 100 percent by weight of the core. The PEG-containing polish coat is preferably applied so as to impart about a 0.25 percent weight gain, based upon 100 percent by weight of the core. The wax layer, if used in the final polish coat, is typically present in an amount of about 1 to about 30 weight percent of the polish coat. This represents about 0.05 to about 1.00 mg per tablet, preferably about 0.2 to about 0.5 mg per tablet. It should be understood however that the above ranges are provided for general guidance only. Different pharmaceutical actives will require additional taste-masking effectiveness. Further, coating levels may be varied to impart different degrees of sweetness, color and polish. Further, although generally not economical, thicker coatings can always be applied.

### DESCRIPTION OF THE PREFERRED EMBODIMENTS

The following examples are intended to describe the present invention without limitation.

### EXAMPLE 1

A coating system as described in Table 1 is prepared as follows. Weight gains expressed below are relative to the core weight.

TABLE 1

| | |
|---|---|
| Subcoat | 2.0% weight gain |
| | 12% solution |
| | 60% by weight of HPMC E5:E15 (2:1 wt. ratio) |
| | 30% by weight of sucrose |
| | 8.8% triacetin |
| | 0.03% propyl gallate |
| | 0.2% acesulfame-K |
| | 1.0% flavoring |
| Colorcoat | 4.0% weight gain |
| | 50% by weight of Opadry ® |
| | 48.8% by weight of sucrose |
| | 0.2% acesulfame-K |
| | 1.0% flavoring |
| Polish Coat | 0.25% weight gain |
| | PEG 8000 |
| | 0.03% Carnauba wax dusting (weight gain) |

(a) Preparation of Subcoat

An appropriate quantity of purified water is weighed out in a stainless steel beaker. Mixing with a shaft-driven, propeller type mixer such as a Lightnin® mixer, available from Mixing Equipment Co., Rochester, N.Y., is initiated. Sucrose is slowly added to the water and mixed until it dissolves. HPMC E5 and HPMC E15 are slowly added to the solution and mixed until the solution is fully hydrated with no visible lumps. Triacetin, propyl gallate, and acesulfame-K are added to the solution and mixed until the solution has no visible lumps.

(b) Colorcoat Preparation

An appropriate quantity of purified water is weighed out in a stainless steel beaker. Mixing with a Lightnin® mixer is initiated. Sucrose is slowly added to the water and mixed until it dissolves. Opadry® Brown (Formulation No. 03B16722) is slowly added to the solution and mixed until the solution is fully hydrated with no visible lumps. Acesulfame-K is added to the solution and mixed until the solution has no visible lumps.

This coating system is coated onto a core of ibuprofen and a disintegrant.

### EXAMPLE 2

The procedure of Example 1 is followed substituting a subcoat as described in Table 2 and a colorcoat as described in Table 3.

TABLE 2
Subcoat

| | Percent by weight based upon |
|---|---|
| Ingredient | 100% by weight of subcoat |
| HPMC E5 | 40.0 |
| Sucrose | 30.0 |
| HPMC E15 | 20.0 |
| Triacetin | 8.8 |
| Propyl Gallate | 0.03 |
| Acesulfame-K | 0.2 |
| Flavoring | 1.0 |

TABLE 3
Colorcoat

| | Percent by weight based upon |
|---|---|
| Ingredient | 100% by weight of colorcoat |
| Opadry ® Brown | 50.0 |
| Sucrose | 48.8 |
| Acesulfame-K | 0.2 |
| Flavoring | 1.0 |

All patents, publications, applications, and test methods mentioned above are hereby incorporated by reference. Many variations of the present matter will suggest themselves to those skilled in the art in light of the above detailed description. All such obvious variations are within the patented scope of the appended claims.

What is claimed is:

1. A coating system for application to solid pharmaceutical dosage units, said system comprising:
a. a subcoat comprising (i) an water soluble cellulosic material and (ii) a sugar component; and
b. a polish coat comprising polyethylene glycol.

2. The coating system, as defined in claim 1, wherein the cellulosic material is selected from the group consisting of hydroxypropyl cellulose, hydroxypropyl methyl cellulose and mixtures thereof.

3. The coating system, as defined in claim 1, wherein the weight ratio of cellulosic material to sugar component within the subcoat ranges from about 40:60 to about 85:15 percent by weight.

4. The coating system, as defined in claim 1, wherein the weight ratio of cellulosic material to sugar component within the subcoat ranges from about 50:50 to about 70:30 percent by weight.

5. The coating system, as defined in claim 2 wherein the hydroxypropyl methyl cellulose is selected from the group consisting of HPMC E5, HPMC E15 and mixtures thereof.

6. The coating system, as defined in claim 5, wherein the weight ratio of hydroxypropyl methyl cellulose to sugar component within the subcoat ranges from about 60:40 to about 70:30 percent by weight.

7. The coating system, as defined in claim 6, wherein the weight ratio of hydroxypropyl methyl cellulose to sugar component within the subcoat is about 66:33 by weight.

8. The coating system, as defined in claim 1, wherein the sugar component comprises a polysaccharide.

9. The coating system, as defined in claim 8, wherein the polysaccharide is sucrose.

10. The coating system, as defined in claim 1, wherein the polish coat comprises a polyethylene glycol selected from those having molecular weights ranging from about 500 to about 50,000, and mixtures thereof.

11. The coating system, as defined in claim 10, wherein the polish coat comprises a polyethylene glycol selected from those having molecular weights ranging from about 4000 to about 15,000, and mixtures thereof.

12. The coating system, as defined in claim 10, wherein the polish coat comprises a polyethylene glycol having a molecular weights of about 8000.

13. The coating system, as defined in claim 1, wherein the polish coat further comprises a wax applied over the surface of the polyethylene glycol-containing layer.

14. The coating system, as defined in claim 13 wherein the wax is Carnauba wax.

15. The coating system, as defined in claim 14, wherein the wax is present in amounts ranging from about 1.0 to 30 wt. percent, based upon the weight of the polish coat.

16. The coating system, as defined in claim 1, further comprising a colorcoat interposed between the subcoat and the polish coat.

17. The coating system, as defined in claim 16, wherein the colorcoat comprises a colorant and at least one sugar component.

18. The coating system, as defined in claim 17, wherein the sugar component is a polysaccharide.

19. The coating system, as defined in claim 18, wherein the polysaccharide is sucrose.

20. The coating system, as defined in claim 17, wherein the sugar component is present in amounts ranging from about 40 to about 60 weight percent of the colorcoat.

21. The coating system, as defined in claim 17, wherein the sugar component is present in amounts of about 50 weight percent of the colorcoat.

22. A composition comprising (i) a pharmaceutically active material, and (ii) an outer coating as defined in claim 1.

23. The composition of claim 22 wherein the pharmaceutically active material is selected from the group consisting of aspirin, acetaminophen, ibuprofen and ketoprofen.

24. A method for preparing a dosage unit form, said method comprising coating a core comprising a medicament and a disintegrant with a coating system as defined in claim 1.

25. A method for producing a glossy finish on a coated solid pharmaceutical dosage unit comprising applying to the surface thereof a polish coat comprising a polyethylene glycol selected from those having molecular weights ranging from about 500 to about 50,000, and mixtures thereof.

26. The method, as defined in claim 25, wherein the polish coat comprises a polyethylene glycol selected from those having molecular weights ranging from about 4000 to about 15,000, and mixtures thereof.

27. The method, as defined in claim 25, wherein the polish coat comprises a polyethylene glycol having a molecular weights of about 8000.

28. The method, as defined in claim 25, wherein the polish coat further comprises a wax applied over the surface of the polyethylene glycol-containing layer.

29. The method, as defined in claim 28 wherein the wax is Carnauba wax.

30. The method, as defined in claim 28, wherein the wax is present in amounts ranging from about 1.0 to 30 wt. percent, based upon the weight of the polish coat.
